(19) United States
(12) Patent Application Publication
(10) Pub. No.: US 2015/0223684 A1
(43) Pub. Date: Aug. 13, 2015

(54) SYSTEM AND METHOD FOR EYE TRACKING

(71) Applicant: BRYSON HINTON, Mesa, AZ (US)

(72) Inventor: BRYSON HINTON, Mesa, AZ (US)

(21) Appl. No.: 14/622,796

(22) Filed: Feb. 13, 2015

Related U.S. Application Data
(60) Provisional application No. 61/939,666, filed on Feb. 13, 2014.

(51) Int. Cl. A61B 3/113; H04N 7/18; G06K 9/00; A61B 3/14

(52) U.S. Cl. CPC A61B 3/113; A61B 3/145; H04N 7/18; G06K 9/00604

(57) ABSTRACT

The present invention comprises a combination of a rotatable screen with a camera module position sensing function coupled with one or more lens and lights to alternatively limit or control the availability of both IR light and visible light to a camera that can be used for both capturing regular video and still photographic images and for eye tracking applications. The most preferred embodiments of the present invention provide for a single camera built into the display scree to be repositioned based on the desired camera functionality and the appropriate light control mechanisms can be activated to achieve the desired result. In a first screen orientation, a first combination of IR and visible lights is activated and in a second screen orientation a second combination of IR and visible lights is activated.

### BACKGROUND OF THE INVENTION

1. Technical Field

The present invention relates generally to technology and more specifically relates to technology used to track eye movement.

2. Background Art

Eye tracking is the process of measuring either the point of gaze (where one is looking) or the motion of an eye relative to the head. An eye tracker is a device for measuring eye positions and eye movement. Eye trackers are used in research on the visual system, in psychology, in cognitive linguistics and in product design. There are a number of methods for measuring eye movement. The most popular variant uses video images from which the eye position is extracted. Other methods use search coils or are based on the electrooculogram.

Eye tracking data is collected using either a remote or head-mounted ‘eye tracker’ connected to a computer. While there are many different types of non-intrusive eye trackers, they generally include two common components: a light source and a camera. The light source (usually infrared) is directed toward the eye. The camera tracks the reflection of the light source along with visible ocular features such as the pupil. This data is used to extrapolate the rotation of the eye and ultimately the direction of gaze. In addition to basic eye position information, other measurements such as blink frequency and changes in the size of the pupil may also be detected by the eye tracker. The aggregated data is typically written to a computerized data file where the data can be analyzed and used for various purposes.

In recent years, the increased sophistication and accessibility of eye tracking technologies have generated a great deal of interest in the commercial sector. Applications include web usability, advertising, sponsorship, package design and automotive engineering. In general, commercial eye tracking applications function by presenting a target stimulus to a sample of consumers while an eye tracker is used to record the activity of the eye.

Examples of target stimuli may include websites, television programs, sporting events, films, commercials, magazines, newspapers, packages, shelf Displays, consumer systems (ATMs, checkout systems, kiosks), and software. The resulting data can be statistically analyzed and graphically rendered to provide evidence of specific visual patterns. By examining fixations, saccades, pupil dilation, blinks and a variety of other behaviors researchers can determine a great deal about the effectiveness of a given medium or product. While some companies complete this type of research internally, there are many private companies that offer eye tracking services and analysis.

One rapidly growing field for eye tracking applications is web usability. While traditional usability techniques are often quite powerful in providing information on clicking and scrolling patterns, eye tracking offers the ability to analyze user interaction between the clicks and how much time a user spends between clicks. This data can provide valuable insight into which features are the most eye-catching, which features cause confusion and which ones are ignored altogether. Specifically, eye tracking can be used to assess search efficiency, branding, online advertisements, navigation usability, overall design and many other site components. Analyses may target a prototype or competitor site in addition to the main client site.

Eye tracking is also commonly used in a variety of different advertising media. Commercials, print ads, online ads and sponsored programs are all conducive to analysis with current eye tracking technology. For instance in newspapers, eye tracking studies can be used to find out in what way advertisements should be mixed with the news in order to catch the subject's eyes. Analyses focus on visibility of a target product or logo in the context of a magazine, newspaper, website, or televised event. This allows researchers to assess in great detail how often a sample of consumers fixates on the target logo, product or ad. In this way, an advertiser can quantify the success of a given campaign in terms of actual visual attention. Another example of this is a study that found that in a search engine results page authorship snippets received more attention than the paid ads or even the first organic result.

Eye tracking is also commonly used in communication systems for disabled persons: allowing the user to speak, send e-mail, browse the Internet and perform other such activities, using only their eyes. Eye control works even when the user has involuntary movement as a result of various muscular disorders or other disabilities, and for those who have glasses or other physical interference which would limit the effectiveness of older eye control systems. Many computers are now offered with optional eye tracking software and hardware that allows the user to control the computer with eye movement.

While the technology associated with eye tracking has evolved over the years, and many improvements have been made, the technology is not without limitations. For example, while many computers include a built in camera, these built in cameras are generally not useful for eye tracking applications because the functional requirements for eye tracking cameras are very different than the functional requirement for most commercially available web cams and the like. Specifically, eye-tracking cameras are typically located at the bottom of the computer screen and are configured to accept infrared (IR) light while blocking visible light. In contrast, most video/photo cameras associated with computers are located at the top of the computer screen and are configured to accept visible light while blocking IR light. These fundamental differences in functionality make many existing computer cameras unsuitable for use in eye tracking applications. Accordingly, without additional improvements in the state of the art for the implementation of eye tracking capabilities for computers and other screen-based devices, the growth and development of eye tracking applications will continue to be suboptimal.

### BRIEF SUMMARY OF THE INVENTION

The present invention comprises a combination of a rotatable screen with a camera module position sensing function coupled with one or more lens and lights to alternatively limit or control the availability of both IR light and visible light to a camera that can be used for both capturing regular video and still photographic images as well as for eye tracking applications. The most preferred embodiments of the present invention provide for a single camera built into the display screen to be repositioned based on the desired camera functionality and the appropriate light control mechanisms can be activated to achieve the desired result. In a first screen orientation, a first combination of IR and visible lights is activated and in a second screen orientation a second combination of IR and visible lights is activated.

### BRIEF DESCRIPTION OF THE FIGURES

The preferred embodiments of the present invention will hereinafter be described in conjunction with the appended drawings, wherein like designations denote like elements, and:

FIG. 1 is a schematic diagram of a rotatable display screen positioned in a first position suitable for use in conjunction with an eye tracking system in accordance with a preferred exemplary embodiment of the present invention;

FIG. 2 is a schematic diagram of a rotatable display screen positioned in a second position suitable for use in conjunction with an eye tracking system in accordance with a preferred exemplary embodiment of the present invention;

FIG. 3 is a schematic diagram of an eye tracking system in accordance with a preferred exemplary embodiment of the present invention;

FIG. 4 is a schematic diagram of a light control mechanisms suitable for use in conjunction with an eye tracking system in accordance with a preferred exemplary embodiment of the present invention; and

FIG. 5 is a flow chart for a method of eye tracking in accordance with a preferred exemplary embodiment of the present invention.

### DETAILED DESCRIPTION

The present invention comprises a combination of a rotatable screen with a camera module position sensing function coupled with one or more lens and lights to alternatively limit or control the availability of both IR light and visible light to a camera that can be used for both capturing regular video and still photographic images as well as for eye tracking applications. The most preferred embodiments of the present invention provide for a single camera built into the display scree to be repositioned based on the desired camera functionality and the appropriate light control mechanisms can be activated to achieve the desired result.

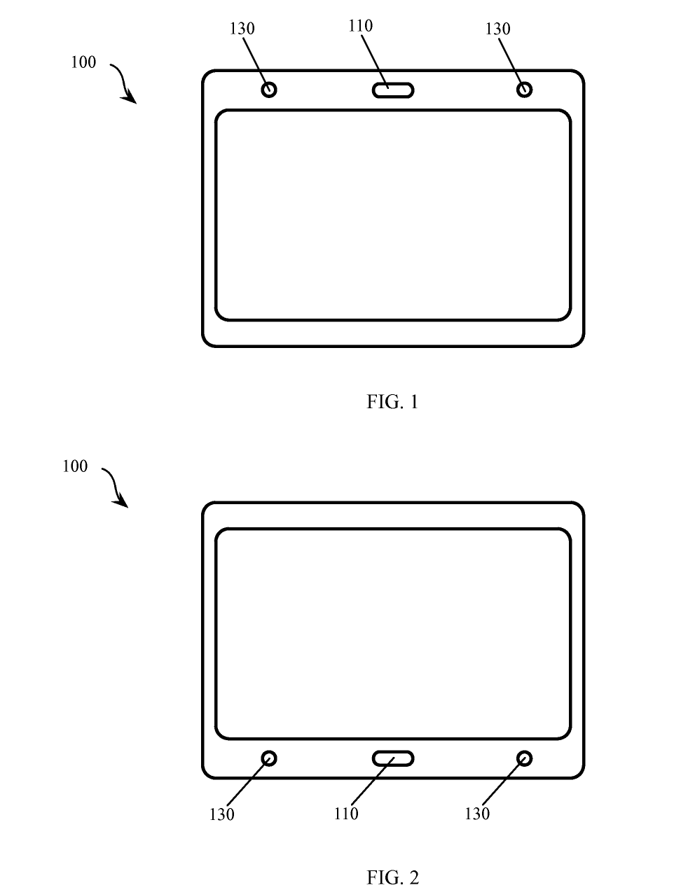

Referring now to FIG. 1, a schematic diagram of a rotatable display screen 100 positioned in a first position suitable for use in conjunction with an eye tracking system in accordance with a preferred exemplary embodiment of the present invention is depicted. For purposes of this disclosure, display screen 100 is any type of display screen, including but not limited to computer screens, tablet screens, smart phone screens, etc. As shown in FIG. 1, screen 100 comprises a camera module 110 and one or more optional IR illuminators 130. With camera module 110 in this position, camera module 110 is positioned and configured to capture standard photo/video images using visible light signals and will block IR light signals via the use of internal IR filters.

In this preferred embodiment of the present invention, the rotation of display screen 100 may be used to trigger a change in the operational characteristics of camera module 110. With camera module 110 located at the top of display screen 100, camera module 110 would be configured to operate in standard photo/video mode, operating much as conventional web cams do. IR illuminators may be present but would not be active in standard photo/video mode.

When in standard photo/video mode, an internal IR filter would be placed over the lens associated with camera module 110. This would block IR light from camera module 110. When in eye tracking mode, an internal visible light-blocking filter would be placed over the lens associated with camera module 110. This would block visible light from camera module 110. The application of the appropriate lens could be automatically triggered based on the orientation of display screen 110 or manually activated by the user via some other electro-mechanical mechanism. A gravity-based approach could also be used where the appropriate filter is positioned by falling into place as display screen 100 is rotated into position. With the appropriate filter in place, camera module 100 would capture the appropriate light signals and perform as expected for the selected application.

Referring now to FIG. 2, a schematic diagram of rotatable display screen 100 positioned in a second position suitable for use in conjunction with an eye tracking system in accordance with a preferred exemplary embodiment of the present invention is depicted. As shown in FIG. 2, screen 100 has been rotated 180° and camera module 110 is now positioned at the bottom of screen 100 and optional IR illuminators 130 are also positioned at the bottom of screen 100. With camera module 110 in this position, camera module 110 is positioned and configured to capture eye tracking images using IR light signals and will block visible light signals.

It should be noted that switching display screen 100 from photo/video mode to eye tracking mode may be triggered by an internal sensor that senses the position of camera module 110 or, alternatively, it may be triggered by a user action such as pressing a physical switch or activating a software based switching mechanism. In either case, the result would be the switching of camera module 110 from photo/video mode to eye tracking mode.

In yet another preferred embodiment of the present invention, an LCD filter is placed over the lens portion of camera module 110. When display screen 100 is positioned for eye tracking mode (e.g., with camera module 110 positioned at the bottom), the LCD filter is activated to block visible light. Similarly, when display screen 100 is positioned for photo/video mode (e.g., with camera module 110 positioned at the top), the LCD filter is deactivated to pass visible light. As explained above, the activation/deactivation could be automatically triggered based on the device orientation or it could be accomplished manually done by the user.

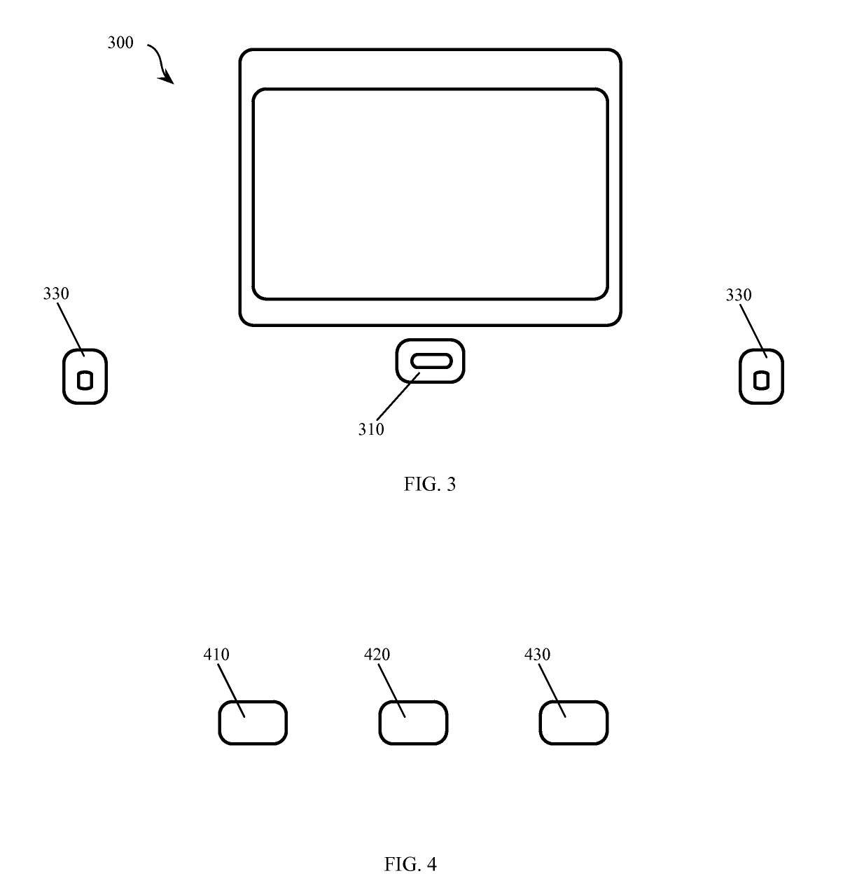

Referring now to FIG. 3, a schematic diagram of an eye tracking system in accordance with a preferred exemplary embodiment of the present invention is depicted. As shown in FIG. 3, a camera module 310 and two IR illuminators are now contained in housings that are separate from display screen 310. This allows camera module 310 and IR illuminators 330 to be positioned in the most optimal positions for eye tracking applications.

One issue with previous eye tracking applications has been the difficulty of accurately tracking eye position and movement when the user is positioned at a significant distance from display screen 310. In previous eye tracking hardware configurations, IR illuminators 330 are contained within the frame or bezel of display screen 310, providing a physical limitation on the distance the user could be positioned at relative to screen 310. In order to accurately track and calculate eye movement, IR illuminators must be spaced at a certain distance apart and as the distance of the user from the display unit increases, the distance between the IR illuminators must also be increased. When all of the components (e.g., camera module and IR illuminators) are contained with the frame or bezel, the distance between the IR illuminators is fixed and cannot be increased and, accordingly, the distance the user can be from display screen 310 is significantly limited.

To overcome this limitation, in at least one preferred embodiment of the present invention, camera module 310 and IR illuminators 330 are contained in separate housings and may be advantageously positioned so as to allow for more accurate detection of eye movement. This is especially useful for applications such as large screen TV viewing where the user is most likely to be located at a distance of 10 feet or more from display screen 310. Additionally, in certain applications, camera module 310 may also be configured with lenses as described herein. It is also possible to leave camera module 310 inside the housing or bezel for display screen 300 and have separate housings for IR illuminators 330.

Referring now to FIG. 4, a schematic diagram of a light control mechanisms suitable for use in conjunction with an eye tracking system in accordance with a preferred exemplary embodiment of the present invention is depicted. Each of an LCD filter 410, an IR filter 420, and a visible light filter 430 may be positioned in front of the lens of a camera module as described herein to accomplish the purposes of the invention as set forth herein.

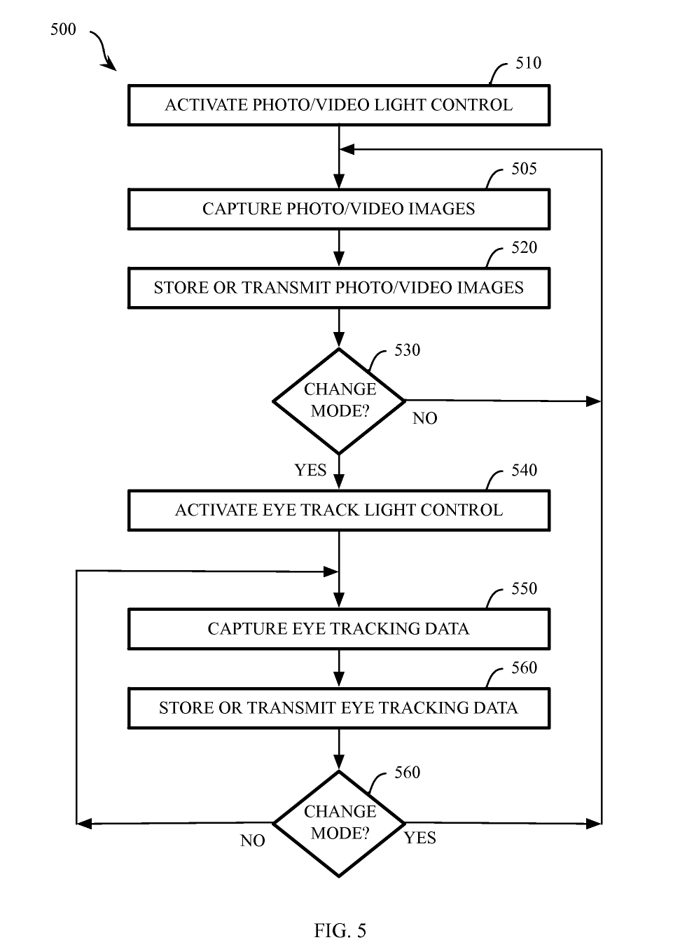

Referring now to FIG. 5, a flow chart for a method of eye tracking in accordance with a preferred exemplary embodiment of the present invention is depicted. For purposes of this figure, the term “light control” refers to the use of one or more lenses and or IR illuminators to interact with an camera module to capture signals for photo/video mode and/or eye tracking mode. As shown in FIG. 5, photo/video light controls for photo/video mode will be activated (step 510). This may include the application of an IR lens filter and/or an LDC filter that has been configured to block IR light signals. This is the initial mode and may be a user configurable default setting or a system-generated setting based on the most frequently used mode, the last used mode, etc.

With the photo/video mode activated, the camera module will capture photo/video images in a fashion that is similar to standard web cams (step 505) and those images will be stored or transmitted as determined by the specific application or applications (step 520).

At some point, the user will desire to switch the camera module from photo/video mode to eye tracking mode and take some action to initiate the switch. This may be a reorientation of the display screen, pressing a button, etc. The camera module will detect the change and if eye-tracking mode has been detected (step 530=“YES”) then the eye tracking light controls will be activated (step 540). This may include the application of a visible light lens filter and/or an LDC filter that has been configured to block visible light signals. If the switch to eye track mode is not detected (step 530=“NO”), then the camera module will remain in photo/video mode.

When operating in eye tracking mode, the camera module will capture eye tracking data (step 550) and store or transmit the eye tracking data as determined by the specific application or applications (step 560). Similar to the switch to eye tracking mode, the camera module will be alerted as to a change from eye tracking mode to photo/video mode (step 560=“YES”) and the process to switch from eye tracking mode to photo/video mode will take place with the appropriate light control mechanisms being activated (step 510). Otherwise (step 560=“NO”), the camera module will remain in eye tracking mode (s

From the foregoing description, it should be appreciated that an effective and efficient for adapting a camera module for use in both eye tracking applications and standard photo/video images is provided by the various preferred embodiments of the present invention and that the various preferred embodiments offer significant benefits that would be apparent to one skilled in the art. Furthermore, while multiple preferred embodiments have been presented in the foregoing description, it should be appreciated that a vast number of variations in the embodiments exist. For example, it should be noted that the exact dimensions and size of the rotatable screen display of the present invention as well as the location of the lenses and IR illuminators described herein in not described so as to limit the preferred embodiments of the invention but provided as exemplary representations for use by those skilled in the art. Other shapes and sizes of screens may be selected; the number and position of the IR illuminators and lenses, etc. may be varied as desired and/or necessary for a specific application.

Accordingly, it should be appreciated that these embodiments are preferred exemplary embodiments only and are not intended to limit the scope, applicability, or configuration of the invention in any way. Rather, the foregoing detailed description provides those skilled in the art with a convenient road map for implementing a preferred exemplary embodiment of the invention, it being understood that various changes may be made in the function and arrangement of elements described in the exemplary preferred embodiment without departing from the spirit and scope of the invention as set forth in the appended claims.

1. An eye tracking system comprising:
a rotatable screen housing;
a camera module; and
at least one light control mechanism, the at least one light control mechanism activating a first combination of light filters when the rotatable screen housing is positioned in a first orientation, the at least one light control mechanism activating a second combination of light filters when the rotatable screen housing is positioned in a second orientation.

2. The eye tracking system of claim 1 wherein the at least one light control mechanism comprises at least a first filter associated with the camera module, the at least a first filter being configured to selectively filter out IR light waves when the rotatable screen housing is positioned in the first orientation.

3. The eye tracking system of claim 1 wherein the at least one light control mechanism comprises at least a first filter associated with the camera module, the at least a first filter being configured to selectively filter out visible light waves when the rotatable screen housing is positioned in the second orientation.

4. The eye tracking system of claim 1 wherein the first combination of light filters comprises at least an IR light filter and a visible light wave light filter.

5. The eye tracking system of claim 1 wherein the second combination of light filters comprises at least an IR light filter and a visible light wave light filter.

6. The eye tracking system of claim 1 wherein the first combination of light filters is housed with the rotatable screen housing and wherein first combination of light filters is positioned so as to be selectively placed over a lens associated with the camera module.

7. The eye tracking system of claim 1 wherein the second combination of light filters is housed with the rotatable screen housing and wherein second combination of light filters is positioned so as to be selectively placed over a lens associated with the camera module.

8. The eye tracking system of claim 1 wherein the first combination of light filters is housed in a housing other than the rotatable screen housing and wherein first combination of light filters is positioned so as to be selectively placed over a lens associated with the camera module.

9. The eye tracking system of claim 1 wherein the second combination of light filters is housed in a housing other than the rotatable screen housing and wherein second combination of light filters is positioned so as to be selectively placed over a lens associated with the camera module.

10. The eye tracking system of claim 1 wherein the second combination of light filters is housed with the rotatable screen housing and wherein second combination of light filters is positioned so as to be selectively placed over a lens associated with the camera module and wherein the first combination of light filters is housed in a housing other than the rotatable screen housing and wherein first combination of light filters is positioned so as to be selectively placed over a lens associated with the camera module.

11. An eye tracking method comprising the steps of:
providing a rotatable screen housing;
providing a camera module; and
providing at least one light control mechanism, the at least one light control mechanism activating a first combination of light filters when the rotatable screen housing is positioned in a first orientation, the at least one light control mechanism activating a second combination of light filters when the rotatable screen housing is positioned in a second orientation.

12. The eye tracking method of claim 11 wherein the at least one light control mechanism comprises at least a first filter associated with the camera module, the at least a first filter being configured to selectively filter out IR light waves when the rotatable screen housing is positioned in the first orientation.

13. The eye tracking method of claim 11 wherein the at least one light control mechanism comprises at least a first filter associated with the camera module, the at least a first filter being configured to selectively filter out visible light waves when the rotatable screen housing is positioned in the second orientation.

14. The eye tracking method of claim 11 wherein the first combination of light filters comprises at least an IR light filter and a visible light wave light filter.

15. The eye tracking method of claim 11 wherein the second combination of light filters comprises at least an IR light filter and a visible light wave light filter.

16. The eye tracking method of claim 11 wherein the first combination of light filters is housed with the rotatable screen housing and wherein first combination of light filters is positioned so as to be selectively placed over a lens associated with the camera module.

17. The eye tracking method of claim 11 wherein the second combination of light filters is housed with the rotatable screen housing and wherein second combination of light filters is positioned so as to be selectively placed over a lens associated with the camera module.

18. The eye tracking method of claim 11 wherein the first combination of light filters is housed in a housing other than the rotatable screen housing and wherein first combination of light filters is positioned so as to be selectively placed over a lens associated with the camera module.

19. The eye tracking method of claim 11 wherein the second combination of light filters is housed in a housing other than the rotatable screen housing and wherein second combination of light filters is positioned so as to be selectively placed over a lens associated with the camera module.

20. The eye tracking method of claim 11 wherein the second combination of light filters is housed with the rotatable screen housing and wherein second combination of light filters is positioned so as to be selectively placed over a lens associated with the camera module and wherein the first combination of light filters is housed in a housing other than the rotatable screen housing and wherein first combination of light filters is positioned so as to be selectively placed over a lens associated with the camera module.
